(19) United States
(12) Patent Application Publication
(10) Pub. No.: US 2026/0084496 A1
(43) Pub. Date: Mar. 26, 2026

(54) FRAGRANCE MODULE FOR VEHICLE

(71) Applicant: Hanon Systems, Daejeon (KR)

(72) Inventors: Changhyun BAEK, Daejeon (KR); Sang Ho OH, Daejeon (KR); Young Chul KIM, Daejeon (KR); Chaegeun LIM, Daejeon (KR); Yun Sung JO, Daejeon (KR)

(73) Assignee: Hanon Systems, Daejeon (KR)

(21) Appl. No.: 19/108,142

(22) PCT Filed: Sep. 27, 2023
(86) PCT No.: PCT/KR2023/015071

§ 371 Date: Feb. 28, 2025

(30) Foreign Application Priority Data
Dec. 23, 2022 (KR) 10-2022-0183487

(51) Int. Cl. B60H 3/00; A61L 9/14; F16H 37/12

(52) U.S. Cl. CPC B60H 3/0035; A61L 9/145; F16H 37/124; A61L 2209/133; A61L 2209/134; A61L 2209/16

(57) ABSTRACT

The present invention relates to a fragrance module for vehicle provided in a vehicle air conditioning system to introduce a fragrance material into a vehicle cabin during the air conditioning of the vehicle cabin, and more specifically, to a fragrance module for vehicle from which a fragrance material is dispensed by pushing a cartridge plunger and that may keep fragrance dispensing intensity constant when the plunger is pushed.

### TECHNICAL FIELD

The present invention relates to a fragrance module for vehicle installed in a vehicle air conditioning system to introduce a fragrance material into a vehicle cabin during the air conditioning of the vehicle cabin, and more particularly, to a fragrance module for vehicle from which a fragrance material is dispensed by pushing a cartridge plunger and that may keep the fragrance dispensing intensity constant when the plunger is pushed.

### BACKGROUND ART

In general, a vehicle air conditioning system is configured to cool or heat a vehicle cabin by selectively passing outside air drawn toward the vehicle cabin by a blower unit through an evaporator where a refrigerant flows or a heater core where a vehicle engine coolant flows for heat exchange and then distributing the air in the state of cold air passing through the evaporator or warm air passing through the heater core to various direction dispensing in the vehicle cabin through a defrost vent, a face vent, and a floor vent connected to each part of the vehicle cabin.

Such a vehicle air conditioning system may introduce various harmful substances such as exhaust gas generated on the road into the vehicle cabin during operation, and when operation is terminated in a state where the condensate on the evaporator is not fully dried, bacterial foreign substances such as mold inhabiting the air conditioning system due to the humid environment may enter the vehicle cabin to cause unpleasant odors in the vehicle cabin.

Fragrance dispensers for purifying the vehicle cabin air by eliminating odors have been recently popularized to address these issues.

Typical fragrance dispensers include a cartridge storing a fragrance material and an actuator driven by the power supply and control signals, wherein the actuator rotates an eccentric shaft and applies pressure to a plunger of the cartridge through the eccentric shaft to dispense the aroma.

In the case of conventional cartridge push-type fragrance dispensers as described above, the inability to precisely control the eccentric shaft and the difficulty in fixing the eccentric shaft in a specific position posed the challenge of keeping the fragrance dispensing intensity constant. In particular, when controlling two or more cartridges with a single eccentric shaft, it was even more challenging to keep the fragrance dispensing intensity constant for each cartridge.

### DISCLOSURE

### Technical Problem

The present invention is devised to address the aforementioned issues, and an object of the present is to provide a fragrance module for vehicle that allows precise control of the fragrance dispensing intensity by arranging the rotation axis of the actuator and the pressing direction of the cartridge plunger in parallel and applying a plate-shaped pressing member between the actuator and the cartridge.

Another object of the present invention is to provide a fragrance module for vehicle that allows keeping the desired fragrance dispensing intensity constant even without precise control of the actuator by providing the plate-shaped pressing member with an inclined portion to control the push distance and providing at least one horizontal section on the inclined portion.

The present invention further aims to provide a fragrance module for vehicle that, when a plurality of cartridges is provided, applies the same push pressure to each cartridge when the pressing member is driven, by arranging the plurality of cartridges radially around the rotation axis of the actuator and respectively arranging the inclined portions of the plate-shaped pressing member at the same distance from the rotation axis of the actuator.

### Technical Solution

The fragrance module for vehicle of the present invention includes an actuator rotating a drive shaft; a drive wheel connected to the drive shaft to convert the rotational force of the drive shaft into a linear pressing motion; a push bar transmitting the linear pressing motion of the drive wheel to a plunger of a cartridge to dispense the fragrance material; and a cartridge storing the fragrance material inside and dispensing the inside fragrance material to the outside when pressure is applied to the plunger, wherein a first axial direction of the drive shaft and a second axial direction, which is the pressing direction of the push bar, are parallel to each other.

At this time, the drive wheel includes a plate-shaped wheel body; a wheel shaft extending outward from one surface of the wheel body and connected to the drive shaft of the actuator; and a protruding inclined portion protruding outward from the other surface of the wheel body and converting the rotational motion of the wheel body into a linear motion through the push bar, wherein the protrusion length of the protruding inclined portion progressively increases from one side of the wheel body to the other side in the in the circumferential direction.

In addition, the protruding inclined portion includes an inclined surface having a protrusion length that progressively increases from one side of the wheel body to the other side in the circumferential direction, and the pressing displacement of the push bar coming into contact with the inclined surface varies according to the rotational displacement of the wheel body.

In addition, the protruding inclined portion further includes a flat section extending to the other side from the end of the inclined surface on the other side in the circumferential direction but extending to a predetermined protrusion length, and when the push bar is positioned on the flat section, the push bar maintains a fixed pressing displacement even when the wheel body rotates.

In addition, the protruding inclined portion further includes a plurality of flat sections formed on the inclined surface but extending to a predetermined protrusion length in the circumferential direction, the plurality of flat sections is arranged on the inclined surface at intervals in the circumferential direction, and when the push bar is positioned on a flat section among the plurality of flat sections, the push bar maintains a fixed pressing displacement even when the wheel body rotates.

In addition, the fragrance module for vehicle includes a plurality of push bars and a plurality of cartridges, wherein a plurality of protruding inclined portions is arranged on the wheel body at intervals in the circumferential direction to control the dispensing of the fragrance material from the plurality of cartridges.

In addition, the radial distance from the drive shaft to each of the plurality of protruding inclined portions is the same.

In addition, the angle formed in the circumferential direction with respect to the center of the wheel shaft by the flat section is equal to the rotational tolerance angle of the actuator or greater than the rotational tolerance angle.

Moreover, the angle formed in the circumferential direction with respect to the center of the wheel shaft by the flat section is equal to or greater than ±2 degrees.

### Advantageous Effects

The fragrance module for vehicle of the present invention with the above configuration is configured such that the rotation axis of the actuator and the push direction of the cartridge plunger are parallel to each other to have the effect that the push displacement of the plunger by the rotation of the actuator may be precisely controlled.

In addition, the push displacement of the plunger is controlled through the inclined portion of the plate-shaped pressing member, but at least one horizontal section is provided on the inclined portion to keep the push displacement of the plunger constant throughout the horizontal section to have the effect that the fragrance dispensing intensity of the cartridge may be kept constant even without precise control of the actuator.

Moreover, when a plurality of cartridges is provided, a plurality of inclined portions provided in the plate-shaped pressing member may be respectively arranged at the same distance as the rotation axis of the actuator to have the effect that the plurality of cartridges may be controlled at the same fragrance dispensing intensity.

### DESCRIPTION OF DRAWINGS

FIG. 1 is a front view of a fragrance module for vehicle according to a first embodiment of the present invention.

FIG. 2 is a perspective view of a fragrance module for vehicle according to the first embodiment of the present invention.

FIG. 3 is a front view of a drive wheel according to the first embodiment of the present invention.

FIG. 4 is a frontal schematic view of a drive wheel according to a 1-1 embodiment of the present invention.

FIG. 5 is a frontal schematic view of a front wheel according to a 1-2 embodiment of the present invention.

FIG. 6 is a frontal schematic view of a drive wheel according to a 1-3 embodiment of the present invention.

FIG. 7 is a perspective view of a fragrance module for vehicle according to a

### SECOND EMBODIMENT OF THE PRESENT INVENTION

FIG. 8 is a plan view of a drive wheel according to the second embodiment of the present invention.

### Mode for Invention

Embodiments of the present invention will be described in detail with reference to the drawings below.

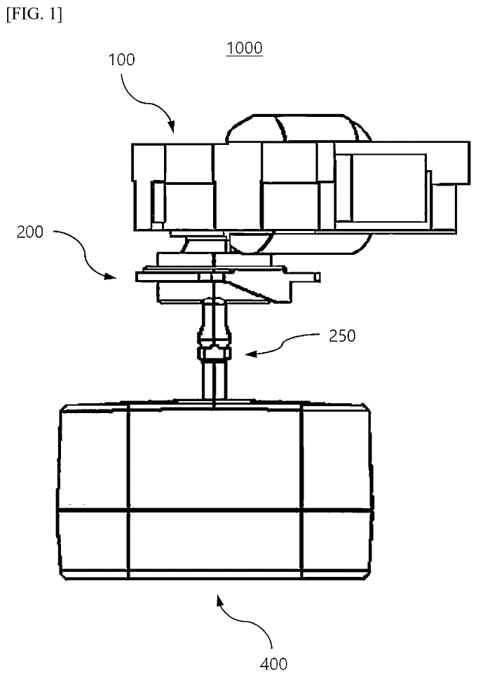

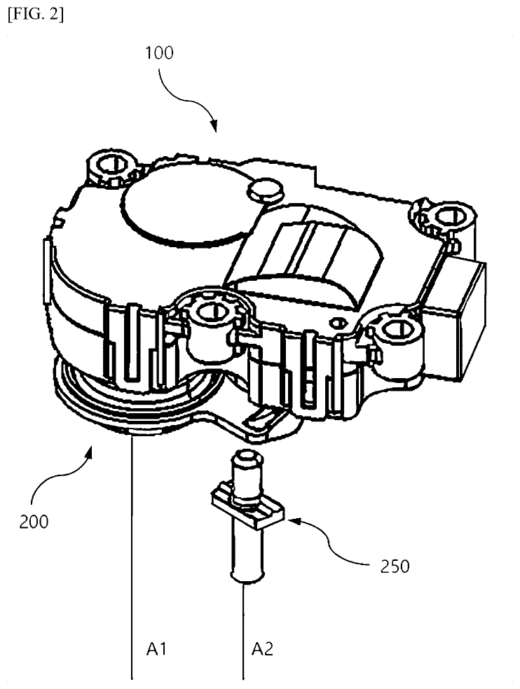

A front view of a fragrance module for vehicle 1000 according to a first embodiment of the present invention is shown in FIG. 1, and a perspective view of the fragrance module for vehicle 1000 according to the first embodiment of the present invention is shown in FIG. 2.

As illustrated, the fragrance module for vehicle 1000 according to an embodiment of the present invention includes an actuator 100 rotating a drive shaft by the power supply and control signals, a drive wheel 200 connected to a drive shaft of the actuator 100 to convert a rotational force of the drive shaft into a linear pressing motion, a push bar 250 transmitting the linear pressing motion of the drive wheel 200 to a plunger of a cartridge 400 to dispense a fragrance material, and a push-type cartridge 400 storing the fragrance material inside and dispensing the inside fragrance material to the outside when pressure is applied to the plunger.

At this time, the fragrance module for vehicle 1000 according to the first embodiment of the present invention is configured such that the first axial direction A1 of the drive shaft of the actuator 100 and the second axial direction A2, which is the pressing direction of the push bar 250 are parallel to each other. Arranging the first axial direction A1 and the second axial direction Al in parallel offers the advantage that the fragrance dispensing intensity may be precisely controlled.

Specific embodiments of the fragrance module for vehicle 1000 having the above features will be described in detail with reference to the drawings.

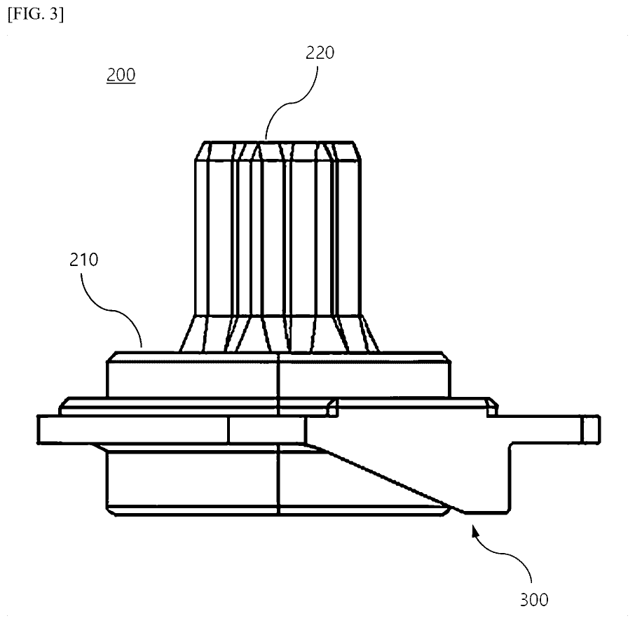

A front view of the drive wheel 200 according to the first embodiment of the present invention is shown in FIG. 3. As illustrated, the drive wheel 200 includes a disc-shaped wheel body 210 having a predetermined thickness, a wheel shaft 220 extending from one side of the wheel body 210 in the one-side direction and connected to the drive shaft of the actuator 100, and a protruding inclined portion 300 extending from the other side of the wheel body 210 in the-other-side direction and converting a rotational motion of the wheel body 210 into a linear motion through a push bar 250. The protruding inclined portion 300 may have the protrusion length that progressively increases from one side of the wheel body 210 to the other side in the circumferential direction and come into contact with the end of the push bar 250 on one side such that when the wheel body 210 rotates, pressure is axially applied to the push bar 250 to cause the push bar 250 to engage in a linear motion in the-other-side direction. At this time, the protruding inclined portion 300 of the present invention may control the linear motion of the push bar 250 more precisely through various embodiments.

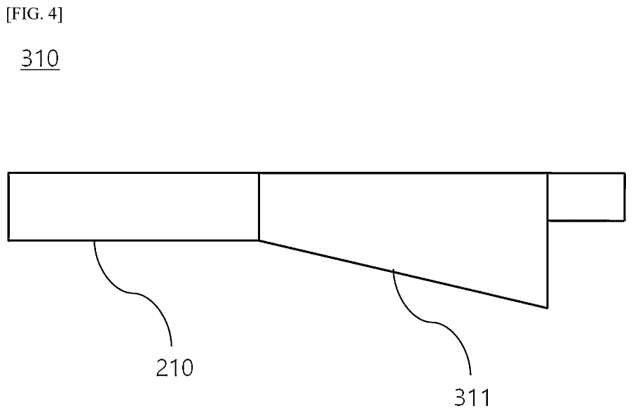

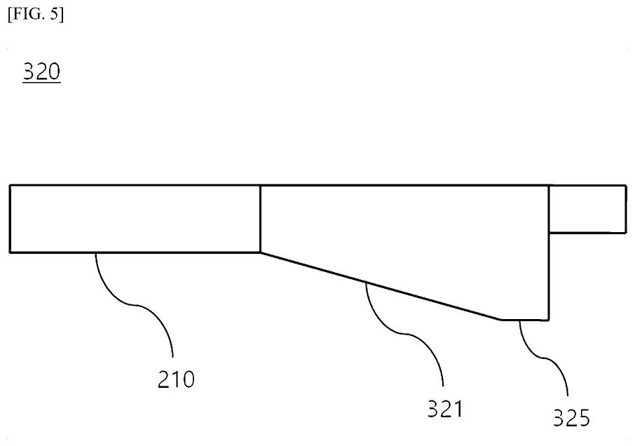

Frontal schematic views of the protruding inclined portion 300 according to various embodiments of the present invention are shown in FIGS. 4 to 6.

FIG. 4 shows that the protruding inclined portion 310 according to a 1-1 embodiment of the present invention may include an inclined surface 311 having a protrusion length that progressively increases from one side of the wheel body 210 to the other side in the circumferential direction. Accordingly, the pressure-applying displacement of the push bar 250 coming into contact with the inclined surface 311 varies according to the rotational displacement of the wheel body 210 to cause the plunger pressing displacement of the cartridge 400 to vary so that the dispensing intensity of the fragrance material may be controlled.

In addition, FIG. 5 shows that the protruding inclined portion 320 according to a 1-2 embodiment of the present invention includes an inclined surface 321 having a protrusion length that progressively increases from one side of the wheel body 210 to the other side in the circumferential direction. At this time, the protruding inclined portion 320 may further include a flat section 325 extending to the other side from the end of the inclined surface 321 on the other side in the circumferential direction but extending to a predetermined protrusion length. Accordingly, the pressing displacement of the plunger varies according to the rotational displacement of the wheel body 210, but when the push bar 250 is positioned on the flat section 325 of the protruding inclined portion 320, the push bar 250 may remain fixed even if the wheel body 210 rotates slightly or vehicle vibrations are transmitted. This offers the advantage that the fragrance dispensing intensity of the cartridge may be kept at a specific intensity even without precise control of the actuator.

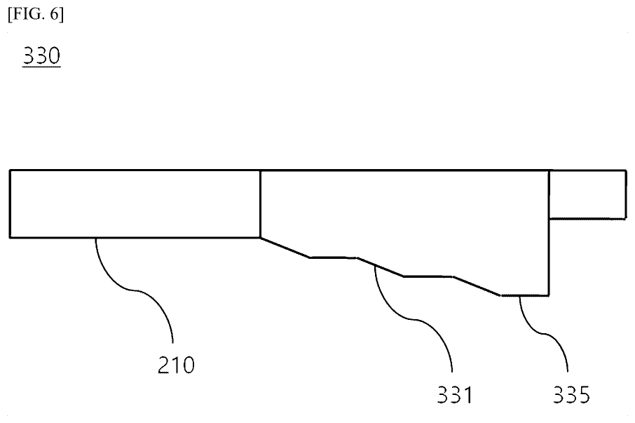

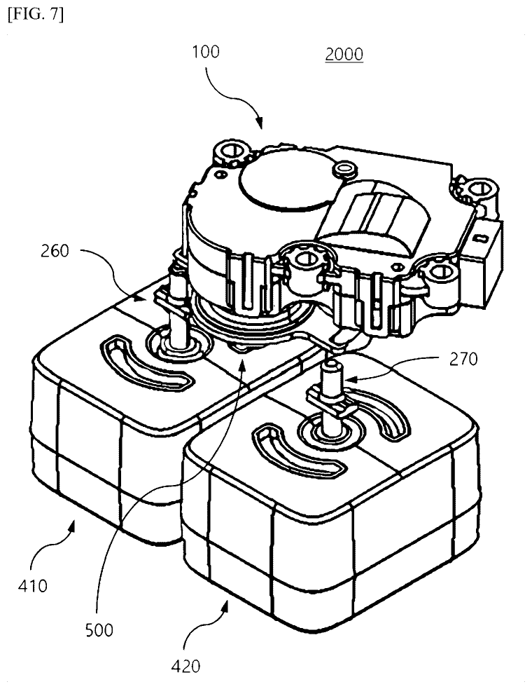

In addition, FIG. 6 shows that a protruding inclined portion 330 according to a 1-3 embodiment of the present disclosure includes an inclined surface 331 having a protrusion length that progressively increases from one side of the wheel body 210 to the other side in the circumferential direction. At this time, the protruding inclined portion 330 may further include a plurality of flat sections 335 extending to the other side from the end of the inclined surface 331 on the other side in the circumferential direction to a predetermined protrusion length. The plurality of flat sections 335 may be arranged at intervals in the circumferential direction of the wheel body 210 on the inclined surface 331. Accordingly, when the pressing displacement of the plunger varies according to the rotational displacement of the wheel body 210 but the push bar 250 is positioned on the flat section 335 of the protruding inclined portion 330, the push bar 250 may remain fixed even if the wheel body 210 rotates slightly. This offers the advantage that the fragrance dispensing intensity of the cartridge may be kept constant even without precise actuator control. In particular, in the 1-3 embodiment, arranging a plurality of flat sections 335 at intervals on the inclined surface 331 offers the advantage that various levels of fragrance dispensing intensity, as well as a specific fragrance dispensing intensity, may be kept constant.

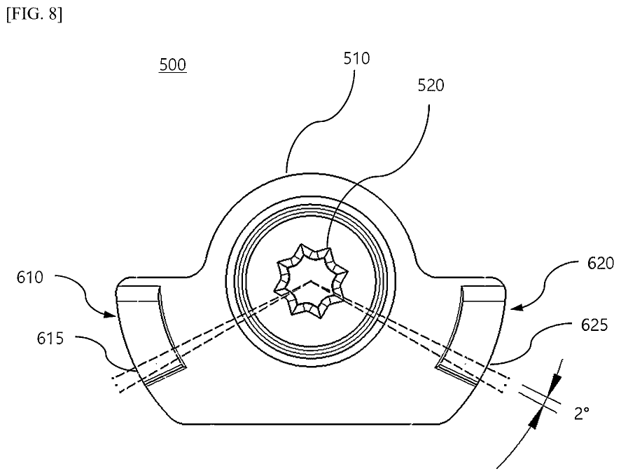

A perspective view of a fragrance module for vehicle 2000 according to a second embodiment of the present invention is shown in FIG. 7, and a plan view of the drive wheel 500 according to the second embodiment of the present invention in FIG. 8.

As illustrated, a fragrance module for vehicle 2000 according to an embodiment of the present invention includes the actuator 100 rotating a drive shaft by the power supply and control signals, a drive wheel 500 connected to the drive shaft of the actuator 100 to convert a rotational force of the drive shaft into a linear pressing motion, first and second push bars 260, 270 transmitting the linear pressing motion of the drive wheel 500 to plungers of the cartridges 410, 420 to dispense fragrance materials, and first and second push-type cartridges 410, 420 storing the fragrance materials and dispensing the inside fragrance material to the outside when pressure is applied to the plunger. The fragrance module for vehicle 2000 according to this embodiment drives the two first and second push bars 260, 270 through a single drive wheel 500 to dispense the fragrance materials from the two first and second cartridges 410, 420.

At this time, a pair of first and second protruding inclined portions 610, 620 provided in the drive wheel 500 may be arranged at an interval in the circumferential direction of the drive wheel 500. More specifically, a first distance from the drive shaft of the actuator 100 to the first protruding inclined portion 610 and a second distance from the drive shaft to the second protruding inclined portion 620 may be equal. Accordingly, the force applied to the first push bar 260 from the drive wheel 500 through the first protruding inclined portion 610 and the force applied to the second push bar 270 from the second protruding inclined portion 620 may be equal. The consequent effect is that the plurality of first and second cartridges 410, 420 may be controlled at the same fragrance dispensing intensity.

FIG. 8 shows the drive wheel 500 includes the wheel body 510 having a predetermined thickness, the wheel shaft 520 extending from one side of the wheel body 510 in the one-side direction and connected to the drive shaft of the actuator 100, and first and second protruding inclined portions 610, 620 protruding from the other side of the wheel body 510 in the-other-side direction and converting the rotational motion of the wheel body 510 into the linear motion through the first and second push bars 260, 270. At this time, the first protruding inclined portion 610 and the second protruding inclined portion 620 may be arranged at an interval in the circumferential direction of the wheel body 510. In addition, the protrusion lengths of the protruding inclined portions 610, 620 progressively increase from one side of the wheel body 510 to the other side in the circumferential direction.

On the other hand, flat sections 615, 625 may be formed at the end of the protruding inclined portions 610, 620 on the other side in the circumferential direction, and the flat sections 615, 625 may have circumferential lengths corresponding to ±2 degrees or more in the circumferential direction with respect to the center of the wheel shaft 520.

This is because the minimum control tolerance of the actuator 100 is about ±2 degrees, so the circumferential length of the flat section must correspond to ±2 degrees or more to have the effect expected of the flat section.

The technical ideas should not be construed as limited to the above-described embodiments of the present invention. The scope of application is diverse, and various modified implementations are possible by those skilled in the art without departing from the essence of the present invention claimed in the Claims. Accordingly, such improvements and alterations fall within the scope of protection of the present invention as long as they are evident to those skilled in the art.

### DESCRIPTION OF DRAWING SYMBOLS

1000, 2000: fragrance module for vehicle
100: actuator
200, 500: drive wheel
210, 510: wheel body
220, 520: wheel shaft
250: push bar
260: first push bar
270: second push bar
300, 310, 320, 330: protruding inclined portion
310, 311, 321, 331: inclined surface
325, 335: flat section
400: cartridge
410: first cartridge
420: second cartridge
610: first protruding inclined portion
620: second protruding inclined portion
615, 625: flat section

1. A fragrance module for vehicle comprising:
an actuator rotating a drive shaft;
a drive wheel connected to the drive shaft to convert a rotational force of the drive shaft into a linear pressing motion;
a push bar transmitting the linear pressing motion of the drive wheel to a plunger of a cartridge to dispense a fragrance material; and
a cartridge storing the fragrance material inside and dispensing the inside fragrance material to the outside when pressure is applied to the plunger, wherein
a first axial direction of the drive shaft and a second axial direction, which is a pressing direction of the push bar, are parallel to each other.

2. The fragrance module for vehicle of claim 1, wherein
the wheel drive includes
a plate-shaped wheel body;
a wheel shaft extending outward from one surface of the wheel body and connected to the drive shaft of the actuator; and
a protruding inclined portion protruding outward from the other surface of the wheel body and converting a rotational motion of the wheel body into a linear motion through the push bar and
the protrusion length of the protruding inclined portion progressively increases from one side of the wheel body to the other side in the circumferential direction.

3. The fragrance module for vehicle of claim 2, wherein
the protruding inclined portion includes an inclined surface having a protrusion length that progressively increases from one side of the wheel body to the other side in the circumferential direction and
the pressing displacement of the push bar coming into contact with the inclined surface varies according to the rotational displacement of the wheel body.

4. The fragrance module for vehicle of claim 3, wherein
the protruding inclined portion further includes a flat section extending to the other side from an end of the inclined surface on the other side in the circumferential direction but extending to a predetermined protrusion length, wherein
when the push bar is positioned on the flat section, the push bar maintains a fixed pressing displacement even when the wheel body rotates.

5. The fragrance module for vehicle of claim 3, wherein
the protruding inclined portion further includes a plurality of flat sections formed on the inclined surface but extending to a predetermined protrusion length in the circumferential direction and
the plurality of flat sections is arranged at intervals in the circumferential direction on the inclined surface, and when the push bar is positioned on a flat section among the plurality of flat sections, the push bar maintains a fixed pressing displacement even when the wheel body rotates.

6. The fragrance module for vehicle of claim 2, comprising a plurality of push bars and a plurality of cartridges, wherein
a plurality of protruding inclined portions is arranged on the wheel body at intervals in the circumferential direction to control the dispensing of the fragrance material from the plurality of cartridges.

7. The fragrance module for vehicle of claim 6, wherein the radial distance from the drive shaft to each of the plurality of protruding inclined portions is the same.

8. The fragrance module for vehicle of claim 4, wherein the angle formed by the flat section in the circumferential direction with respect to the center of the wheel shaft is equal to a rotational tolerance angle of the actuator or greater than the rotational tolerance.

9. The fragrance module for vehicle of claim 8, wherein the angle formed by the flat section in the circumferential direction with respect to the center of the wheel shaft by the flat section is equal to or greater than ±2 degrees.

10. The fragrance module for vehicle of claim 5, wherein the angle formed by the flat section in the circumferential direction with respect to the center of the wheel shaft is equal to a rotational tolerance angle of the actuator or greater than the rotational tolerance.
